(19) United States
(12) Patent Application Publication
(10) Pub. No.: US 2024/0310465 A1
(43) Pub. Date: Sep. 19, 2024

(54) METHOD AND DEVICE FOR DETECTING SUBCLINICAL HYPOXEMIA USING WHOLE BLOOD T2p

(71) Applicant: Texas Tech University System, Lubbock, TX (US)

(72) Inventor: David P. Cistola, El Paso, TX (US)

(73) Assignee: Texas Tech University System, Lubbock, TX (US)

(21) Appl. No.: 18/608,144

(22) Filed: Mar. 18, 2024

Related U.S. Application Data
(60) Provisional application No. 63/452,984, filed on Mar. 17, 2023.

(51) Int. Cl. G01N 33/72

(52) U.S. Cl. CPC G01R 33/50; G01N 33/4925; G01N 33/726; G01R 33/465

(57) ABSTRACT

Embodiments of the present disclosure pertain to methods of detecting hypoxemia in a subject by: (1) receiving a blood sample from the subject; (2) measuring the T₂ relaxation time constant of the blood cell (e.g., blood cell pellet) of the blood sample (T_{2P} value); and (3) correlating the measured T_{2P} value to hypoxemia. In some embodiments, the methods of the present disclosure also include a step of correlating the measured T_{2P} value to the subject's susceptibility to one or more hypoxemia-related conditions. Further embodiments of the present disclosure pertain to systems for detecting hypoxemia in a subject in accordance with the methods of the present disclosure.

### CROSS-REFERENCE TO RELATED APPLICATIONS

This application claims priority to U.S. Provisional Patent Application No. 63/452,984, filed on Mar. 17, 2023. The entirety of the aforementioned patent application is incorporated herein by reference.

### BACKGROUND

A need exists for more sensitive and readily accessible screening tools for detecting hypoxemia, especially subclinical hypoxemia. Numerous embodiments of the present disclosure aim to address the aforementioned need.

### SUMMARY

In some embodiments, the present disclosure pertains to methods of detecting hypoxemia in a subject by: (1) receiving a blood sample from the subject; (2) measuring the T₂ relaxation time constant of the blood cell of a blood sample (e.g., blood cell pellet of a settled, anti-coagulated whole blood sample) (T_{2P} value); and (3) correlating the measured T_{2P} value to hypoxemia. In some embodiments, the methods of the present disclosure also include a step of correlating the measured T_{2P} value to the subject's susceptibility to one or more hypoxemia-related conditions. In some embodiments, the methods of the present disclosure also include a step of making a treatment decision, such as monitoring the subject and/or administering a therapeutic agent and/or intervention to the subject. In some embodiments, the methods of the present disclosure are repeated after implementing the treatment decision.

Additional embodiments of the present disclosure pertain to systems for detecting hypoxemia in a subject. In some embodiments, the systems of the present disclosure include one or more computer-readable storage mediums having a program code embodied therewith. In some embodiments, the program code includes programming instructions for: (1) receiving a blood sample from the subject; (3) measuring the T₂ relaxation time constant of blood cells of the blood sample (T_{2P} value); and (4) correlating the measured T_{2P} value to hypoxemia. In some embodiments, the systems of the present disclosure also include programming instructions for correlating the measured T_{2P} value to the subject's susceptibility to one or more hypoxemia-related conditions. In some embodiments, the systems of the present disclosure also include programming instructions for recommending a treatment decision. In some embodiments, the systems of the present disclosure also include programming instructions for repeating the programming instructions after the implementation of the treatment decision.

Additional embodiments of the present disclosure pertain to magnetic resonance devices. In some embodiments, the magnetic resonance device is optimized for measuring whole blood T_{2P} values.

### DESCRIPTION OF THE DRAWINGS

FIG. 1A illustrates a method of detecting hypoxemia in a subject.

FIG. 1B illustrates a system for detecting hypoxemia in a subject.

FIGS. 2A-2B show samples of a Carr-Purcell-Meiboom-Gill (CPMG) decay curve and a T₂ profile. FIG. 2A shows the raw CPMG decay curve acquired from a sample of settled, whole human blood using the CPMG pulse sequence. FIG. 2B shows the T₂ profile obtained from an inverse Laplace transformation (ILT) of the decay curve using the CONTIN algorithm. The X-axes in both the decay curve and ILT are time and T2, respectively, in milliseconds.

FIG. 3 shows blood sample T2P (T₂ relaxation time constant of the blood cells of the blood sample) and T_{2S} (T₂ relaxation time constant of the plasma of the blood sample) levels as a function of time. After gentle mixing, a whole blood sample was allowed to sediment spontaneously while collecting a CPMG decay curve every five minutes. The decay curve was analyzed by inverse Laplace transformation. The T₂ data are plotted against settling time. After approximately 40 minutes of spontaneous sedimentation, both T₂ values reached equilibrium.

FIG. 4 shows the frequency distribution and variation of T₂ values observed for 44 apparently healthy human subjects (bars). The mean ±S.D. for T_{2P} was 248.1±20.2 msec, and T_{2S} was 718.6±60.4 msec.

FIG. 5 shows magnetic field gradient formation in the presence of a paramagnetic pellet. Non-functional oxidized hemoglobin (methemoglobin) and reduced deoxy-hemoglobin contain unpaired electrons and thus, are paramagnetic. Reduced oxy-hemoglobin, the functional form in arterial blood that delivers oxygen to tissues, is not paramagnetic. Horizontal lines are magnetic field lines.

FIG. 6 shows the effect of hematocrit on Tes and T_{2P}, showing the r⁻⁶ distance dependence of T_{2S} on hematocrit arising from paramagnetic relaxation enhancement. As the relative height of the plasma supernatant decreases as the hematocrit increases (top right), the plasma water molecules are, on average, located closer to the paramagnetic cell pellet. Thus, they are affected more by paramagnetic relaxation enhancement, lowering T_{2S}. In this ex vivo experiment, the non-physiological high and low values of hematocrit were achieved by removing or adding plasma to the whole blood sample, respectively.

FIG. 7 shows the dependence of signal intensity from T₂-weighted images on hematocrit, derived from benchtop MRI analysis of settled whole blood.

FIG. 8 shows correlation coefficients (Huber M-values) for T_{2P} and biomarkers of cardiometabolic health.

FIG. 9 illustrates a hypothetical mechanistic link whereby hemoglobin oxidation (subclinical met-hemoglobinemia) mediates the association between hyperinsulinemia and downstream hypoxia and chronic inflammation.

FIG. 10 shows causal mediation analysis to test the hypothesis in FIG. 9, using four (4) linked regression equations.

FIG. 11 shows a schematized representation of the results reported in FIG. 10.

FIG. 12 shows results from an ex vivo experiment where a sample of whole human blood was titrated with aqueous sodium nitrite, a pro-oxidant. Here, T_{2P} decreased with increasing amounts of oxidized hemoglobin (% MetHb). Percent MetHb was measured using a blood gas analyzer. By contrast, T_{2S} measured in the same sample did not change with % MetHb. The slope (beta) of the linear least squares regression line for T_{2P} was statistically different from zero (p<0.0001), whereas the slope for the T_{2S} line was statistically indistinguishable from zero (p=0.172). Sodium nitrite is chemically related to nitric acid, the pro-oxidant naturally produced in the human body when blood insulin rises. In chronic hyperinsulinemia, nitric acid may be overproduced, leading to an increase in % Met Hb and a decrease in T_{2P}.

FIGS. 13A-13C show non-limiting examples of benchtop, portable or miniaturized nuclear magnetic resonance (NMR) relaxometry devices that can measure T_{2P}. FIG. 13A shows a Bruker mq20 benchtop time-domain NMR relaxometry instrument operating at 0.47 Tesla (20 MHz for proton). FIG. 13B shows a Resonance Systems benchtop NMR time-domain relaxometry instrument operating at 0.5 Tesla (23 MHz for proton). FIG. 13C shows a Waveguide Corporation miniaturized portable time-domain relaxometry device operating at 0.47 Tesla (20 MHz for proton).

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials define a term in a manner that contradicts the definition of that term in this application, this application controls.

Hypoxemia refers to decreased levels of oxygen in the blood and is typically caused by blocked airways or serious lung, heart or blood diseases. Specific examples are the hypoxemia that occurs in patients with chronic obstructive pulmonary disease (COPD) or in hospitalized patients with COVID-19. In critical care units, the patient's blood oxygen levels are monitored using a combination of pulse oximetry and arterial blood gas analysis.

In contrast with the overt clinical hypoxemia seen in sick patients, a more subtle hidden condition (subclinical hypoxemia) can occur in apparently healthy individuals. The impact of subclinical hypoxemia depends on its intensity and duration, and on the body's ability to compensate for it, both acutely and chronically.

Thus, subclinical hypoxemia does not necessarily mean that cells and tissues are being deprived of oxygen (hypoxia). In fact, subclinical hypoxemia is an under-recognized condition, even though many patients undergoing a wellness exam in a medical office are screened for oxygen saturation using a fingertip pulse oximeter.

Pulse oximetry is a helpful tool for detecting undiagnosed clinical hypoxemia (e.g., undiagnosed lung, heart, blood or airway disease). A resting oxygen saturation from pulse oximetry (SpO₂) below ˜95% at sea level during routine screening would alert the clinician to the possibility of a medical problem and would prompt further evaluation.

However, subtle subclinical hypoxemia is more difficult to detect, as there is no consensus in the medical literature on what constitutes a normal SpO₂ level. For example, a resting SpO₂ of 95% in one patient may be normal. By contrast, a resting SpO₂ of 95% in a different patient would be abnormal if that patient previously had a SpO₂ of 98% under the same conditions. Thus, subtle decreases in the percent of hemoglobin oxygen saturation can elude detection by fingertip pulse oximetry.

Moreover, pulse oximeters do not detect oxidized hemoglobin, also known as methemoglobin or MetHb. Such a limitation is significant because MetHb is non-functional (i.e., it cannot bind and deliver oxygen to the tissues). Levels of MetHb above ˜20% of total hemoglobin produce overt symptoms of hypoxemia while levels above 50% are life-threatening or fatal.

The body has reductase enzymes to convert oxidized MetHb back to reduced, functional Hb. Under normal conditions, these reductases keep the MetHb level in the blood low (i.e., ˜1% on average). However, the reductase system may become overwhelmed under conditions of acute or chronic oxidative stress.

One possible source of oxidative stress is hyperinsulinemia (i.e., high levels of fasting insulin in the blood of individuals with early insulin resistance). Normally, insulin increases the production of nitric oxide, a potent vasodilator that increases blood flow to tissues to aid in nutrient delivery following a meal. However, abnormal chronic elevations in insulin and nitric oxide can lead to oxidative stress, as nitric oxide is a pro-oxidant. To alleviate that stress, the body reduces nitric oxide at the cost of oxidizing hemoglobin and generating more MetHb. In this way, hidden metabolic imbalances could lead to undetected subclinical hypoxemia in apparently healthy individuals.

Over time, subclinical hypoxemia may slowly deprive cells and tissues of oxygen, thereby damaging the beta cells of the pancreas and the inner walls of arteries. Slow loss of beta cells leads to type 2 diabetes, and the progressive blockage of arteries leads to heart disease and stroke.

As such, untreated subclinical hypoxemia can have various health repercussions, such as the onset of diabetes. The diabetes pandemic is a major threat to the health and well-being of many societies. For instance, approximately 14% of the US adult population has diagnosed or undiagnosed diabetes, and the prevalence is projected to double by 2050. The best strategy for altering this trend is to prevent or delay the onset of disease.

Evidence-based prevention programs for type 2 diabetes rely on fasting glucose, hemoglobin Ale (glycated hemoglobin) and/or glucose tolerance tests to identify individuals at increased risk. However, by the time an individual develops glucose intolerance, a 50-70% decline in pancreatic insulin secretory capacity has already occurred. Moreover, glucose and Hb_{A1c} fail to identify early metabolic imbalance, which includes compensated insulin resistance, oxidative stress and inflammation, with subclinical alterations in proteins and lipoproteins in the blood.

Arterial blood gas analysis can be used to detect subclinical abnormalities of hemoglobin, including decreased oxygen saturation and increased oxidized hemoglobin. However, arterial blood gas analysis is expensive and invasive. As such, arterial blood gas analysis is typically used only in critical care units and emergency departments, thereby making the analysis impractical for routine health screening in outpatient clinics.

Accordingly, a need exists for more sensitive and readily accessible screening tools for detecting markers of cardiometabolic health. Numerous embodiments of the present disclosure aim to address the aforementioned need.

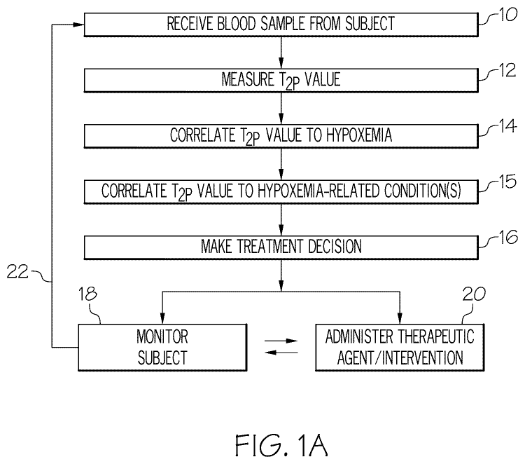

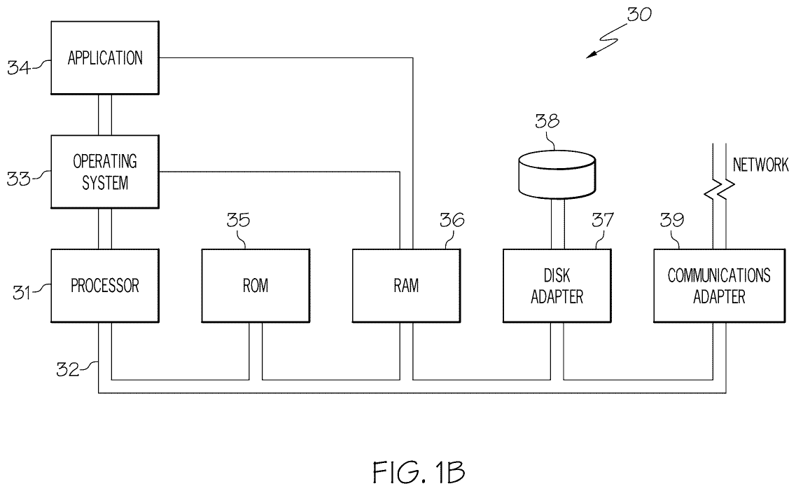

In some embodiments, the present disclosure pertains to methods of detecting hypoxemia in a subject. In some embodiments illustrated in FIG. 1A, the methods of the present disclosure include: receiving a blood sample from the subject (step 10); measuring the T₂ relaxation time constant of the blood cells of the blood sample (e.g., the T₂ relaxation time constant of the blood cell pellet in an anticoagulated sample of settled whole blood) (T_{2P} value) (step 12); and correlating the measured T_{2P} value to hypoxemia (step 14). In some embodiments, the methods of the present disclosure also include a step of correlating the measured T_{2P} value to the subject's susceptibility to one or more hypoxemia-related conditions (step 15). In some embodiments, the methods of the present disclosure also include a step of making a treatment decision (step 16), such as monitoring the subject (step 18) and/or administering a therapeutic agent and/or intervention to the subject (step 20). In some embodiments, the methods of the present disclosure are repeated after implementing the treatment decision (step 22).

In some embodiments, the present disclosure pertains to systems for detecting hypoxemia in a subject. In some embodiments, the systems of the present disclosure include one or more computer-readable storage mediums having a program code embodied therewith. In some embodiments, the program code includes programming instructions for: receiving a blood sample from the subject; measuring the T₂ relaxation time constant of blood cells of the blood sample (T_{2P} value); and correlating the measured T_{2P} value to hypoxemia. In some embodiments, the systems of the present disclosure also include programming instructions for correlating the measured T_{2P} value to the subject's susceptibility to one or more hypoxemia-related conditions. In some embodiments, the systems of the present disclosure also include programming instructions for recommending a treatment decision. In some embodiments, the systems of the present disclosure also include programming instructions for repeating the programming instructions after the implementation of the treatment decision.

As set forth in more detail herein, the methods and systems of the present disclosure can have numerous embodiments.

### Blood Samples

The methods and systems of the present may evaluate various types of blood samples. For instance, in some embodiments, the blood sample includes a whole blood sample. In some embodiments, the whole blood sample contains an anti-coagulant to prevent the blood from clotting.

In some embodiments, the blood sample includes a blood cell component of the blood sample. In some embodiments, the blood cell component includes red blood cells, white blood cells, and platelets. In some embodiments, the blood cell component is mostly comprised of red blood cells, but also white blood cells and platelets.

In some embodiments, the blood cell component is in the form of a pellet. In some embodiments, the pelleted blood cell component is settled or separated from the liquid plasma (i.e., the non-cellular liquid component of the blood) in an anti-coagulated sample of whole blood. In some embodiments, the pelleted blood cell component is in the form of a separated pellet formed by centrifuging the sample, or by spontaneous settling of the cells to the bottom of the tube. In some embodiments, such a process separates the cells from the supernatant plasma. In some embodiments, the settled or separated blood cell component of the blood sample is in anti-coagulated form.

In some embodiments, the blood cell component is in the form of a pellet of an anti-coagulated whole blood sample. In some embodiments, the blood cell pellet represents a settled and anti-coagulated whole blood sample.

In some embodiments, the blood cell component is purified and isolated from the plasma. In some embodiments, the red blood cell component is purified and isolated from the white blood cell, platelet and plasma components. In some embodiments, the blood sample includes a pelleted blood cell component that is settled or separated from the liquid plasma in an anti-coagulated sample of whole blood.

In some embodiments, the methods of the present disclosure also include a step of obtaining a blood sample from a subject. For instance, in some embodiments, a blood sample is obtained from a subject through venipuncture. In some embodiments, a blood sample is obtained from a subject through a fingerstick drop or an upper arm blood collection device equipped with a microtainer tube.

### T_{2P} Values

The methods and systems of the present disclosure may measure various T_{2P} values in various manners. For instance, in some embodiments, the T_{2P} value represents the spin-spin relaxation time constant of the whole blood sample. In some embodiments, the T_{2P} value represents the spin-spin relaxation time constant of the settled or separated blood cell component of the blood sample. In some embodiments, the settled or separated blood cell component of the blood sample is in anti-coagulated form. In some embodiments, the T_{2P} value represents the spin-spin relaxation time constant of the settled blood cell component of the blood sample. In some embodiments, the settled blood cell component of the blood sample is in the form of a cell pellet. In some embodiments, the T_{2P} value represents the spin-spin relaxation time constant of a blood cell component that is in the form of a pellet of an anti-coagulated whole blood sample.

In some embodiments, the T_{2P} value is represented in the following formula for a T₂ relaxation decay curve:

$$I ( t ) = {\sum }_{i} {A}_{i} {e}^{- t / {T}_{2 i}}$$

In some embodiments, I(t) represents the nuclear magnetic resonance (NMR) signal intensity, Aᵢ represents signal amplitude, and T₂ᵢ represents the transverse relaxation time constant of ith proton microenvironment or mobility domain. In some embodiments, when i=P, the proton microenvironment being measured is the blood cell pellet in an anti-coagulated, sedimented whole blood sample. As red blood cells outnumber white blood cells and platelets by ˜1000:1, the dominant contributor to T_{2P} is the water inside of red blood cells. In some embodiments, the value of T_{2P} is influenced by water interactions with hemoglobin, as T_{2P} probes variations in the concentration, oxygenation and oxidation states of hemoglobin. In some embodiments, the T_{2P} term is resolved from other terms using a suitable multi-exponential fitting algorithm, such as an inverse Laplace transformation or matrix pencil method.

T_{2P} values may be measured in various manners. For instance, in some embodiments, T_{2P} values may be measured through the utilization of a magnetic resonance device, a magnetic resonance relaxometry device, a table-top magnetic resonance device, a miniaturized magnetic resonance device, a benchtop magnetic resonance device, a time-domain magnetic resonance device, a magnetic resonance spectroscopy device, a magnetic resonance imaging device, a nuclear magnetic resonance device, or combinations thereof.

### Correlating Measured T_{2P} Value to Hypoxemia

The methods and systems of the present disclosure may correlate measured T_{2P} values to hypoxemia in various manners. For instance, in some embodiments, the correlation includes correlating a lower than average T_{2P} value to hypoxemia. In some embodiments, the lower than average T_{2P} value includes a T_{2P} value lower than 270 ms. In some embodiments, the lower than average T_{2P} value includes a T_{2P} value lower than 250 ms. In some embodiments, the lower than average T_{2P} value includes a T_{2P} value lower than 230 ms. In some embodiments, the lower than average T_{2P} value includes a T_{2P} value lower than 200 ms. In some embodiments, the lower than average T_{2P} value includes a T_{2P} value lower than 180 ms. In some embodiments, the lower than average T_{2P} value includes a T_{2P} value lower than 160 ms. In some embodiments, the lower than average T_{2P} value includes a T_{2P} value lower than 140 ms.

In some embodiments, the lower-than-average T_{2P} value is correlated to hypoxemia by referring to results from a human observational or case-control study, where the cases of hypoxemia (e.g., established by blood gas analysis) have T_{2P} values lower than controls without hypoxemia. In some embodiments, the case-control data can be used to calibrate the optimal T_{2P} cutpoint for detecting hypoxemia, as well as the sensitivity, specificity, and positive and negative predictive values.

In some embodiments, the lower-than-average T_{2P} value is correlated to hypoxemia by referring to regression equations that predict hypoxemia or values of its markers (e.g., percent MetHb or percent deoxy-Hb) from a measured T_{2P} value. In some embodiments, the regression equations are established from a study of human subjects with varying degrees of hypoxemia.

In some embodiments, the lower-than-average T_{2P} value is correlated to hypoxemia through multi-variable regression analysis of human research data, leading to equations that mathematically predict hypoxemia or its markers (percent MetHb or percent deoxy-hemoglobin) from measured T_{2P} values. In some embodiments, the regression equations are established using conventional parametric or non-parametric statistical regression analysis. In some embodiments, the regression analysis is performed using machine-learning or artificial intelligence algorithms.

In some embodiments, a lower-than-average T_{2P} value is attributed to decreased oxygenation of hemoglobin. In some embodiments, a lower-than-average T_{2P} value is attributed to increased oxidation of hemoglobin. In some embodiments, the lower-than-average T_{2P} value is attributed to elevated levels of deoxyhemoglobin (deoxy-Hb). In some embodiments, the lower-than-average T_{2P} value is attributed to elevated levels of oxidized hemoglobin (MetHb). In some embodiments, the lower-than-average T_{2P} value is attributed to elevated levels of both MetHb and deoxy-Hb.

### Correlating Measured T_{2P} Value to Hypoxemia-Related Conditions

In some embodiments, the methods and systems of the present disclosure also include steps or programming instructions for correlating a measured T_{2P} value to a subject's susceptibility to one or more hypoxemia-related conditions. For instance, in some embodiments, a lower-than-average T_{2P} value is correlated to a subject's susceptibility to one or more hypoxemia-related conditions.

Measured T_{2P} values may be correlated to various hypoxemia-related conditions. For instance, in some embodiments, the hypoxemia-related conditions include, without limitation, hypoxia, cellular damage, tissue damage, type 2 diabetes, cardiovascular disease, hyperinsulinemia, insulin resistance, inflammation, oxidative stress, dyslipidemia, hyperglycemia, early metabolic imbalance, prediabetes, metabolic syndrome, adiposity, or combinations thereof.

### Treatment Decisions

In some embodiments, the methods and systems of the present disclosure also include a step or programming instructions for making a treatment decision. In some embodiments, the treatment decision is based on the measured T_{2P} value. In some embodiments, the methods and systems of the present disclosure also include steps or programming instructions for repeating hypoxia detection after implementing the treatment decision. In some embodiments, the methods of the present disclosure also include a step of implementing the treatment decision.

The methods and systems of the present disclosure may make various treatment decisions. For instance, in some embodiments, the treatment decision includes monitoring the subject for signs or symptoms of hypoxemia, administering a therapeutic agent to the subject, or combinations thereof. In some embodiments, the treatment decision includes monitoring the subject for signs or symptoms of hypoxemia. In some embodiments, the treatment decision includes monitoring the subject for signs or symptoms of hypoxemia-related conditions. In some embodiments, the hypoxemia-related conditions include, without limitation, hypoxia, cellular damage, tissue damage, type 2 diabetes, cardiovascular disease, hyperinsulinemia, insulin resistance, inflammation, oxidative stress, dyslipidemia, hyperglycemia, early metabolic imbalance, prediabetes, metabolic syndrome, adiposity, or combinations thereof.

In some embodiments, the treatment decision includes administering a therapeutic agent to the subject. In some embodiments, the treatment decision includes administering an intervention program for the subject. In some embodiments, the intervention program includes, without limitation, a nutritional program, a physical activity program, a non-pharmaceutical intervention, or combinations thereof.

### Subjects

The methods and systems of the present disclosure may be utilized to detect hypoxemia in various subjects. For instance, in some embodiments, the subject is a human being. In some embodiments, the subject is a healthy subject. In some embodiments, the subject is not suffering from or diagnosed with hypoxemia. In some embodiments, the subject shows no hypoxemia-related symptoms.

In some embodiments, the subject suffers from hypoxemia. In some embodiments, the hypoxemia includes sub-clinical hypoxemia. In some embodiments, the hypoxemia includes clinical hypoxemia.

### Operation Methods

The methods of the present disclosure can occur in various manners. For instance, in some embodiments, the methods of the present disclosure occur manually.

In some embodiments, the methods of the present disclosure occur through the utilization of a computer program. In some embodiments, the computer program includes a web-based program, an application-based program, or combinations thereof. In some embodiments, the computer program implements a multi-exponential analysis to extract the T_{2P} value from the raw multi-exponential decay curve. In some embodiments, the computer program implements an inverse Laplace transform algorithm or a matrix pencil algorithm for multi-exponential analysis.

In some embodiments, the computer program includes a machine-learning or artificial intelligence algorithm. In some embodiments, the machine learning or artificial intelligence algorithm is trained on the T_{2P} values and correlated measurements (e.g., correlated health markers). In some embodiments, the training of the machine learning algorithm is conducted using a random forest or bootstrap forest procedure to assess the correlation between T_{2P} and health markers.

In some embodiments, the machine learning or artificial intelligence algorithm is an L1-regularized logistic regression algorithm. In some embodiments, the machine learning or artificial intelligence algorithm includes supervised learning algorithms. In some embodiments, the supervised learning algorithms include nearest neighbor algorithms, naïve-Bayes algorithms, decision tree algorithms, linear regression algorithms, support vector machines, neural networks, convolutional neural networks, ensembles (e.g., random forests and gradient-boosted decision trees), or combinations thereof.

### Systems

The systems of the present disclosure may have various architectures and forms. For instance, in some embodiments, the systems of the present disclosure are in the form of a web-based program, an application-based program, or combinations thereof. In some embodiments, the systems of the present disclosure include a machine-learning algorithm or artificial intelligence algorithm. In some embodiments, the machine learning algorithm or artificial intelligence algorithm is trained on the T_{2P} values and correlated measurements (e.g., correlated health markers).

The systems of the present disclosure can include various types of computer-readable storage mediums. For instance, in some embodiments, the computer-readable storage medium can be a tangible device that can retain and store instructions for use by an instruction execution device. In some embodiments, the computer-readable storage medium may include, without limitation, an electronic storage device, a magnetic storage device, an optical storage device, an electromagnetic storage device, a semiconductor storage device, or combinations thereof. A non-exhaustive list of more specific examples of suitable computer-readable storage medium includes, without limitation, a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device, or combinations thereof.

A computer-readable storage medium, as used herein, is not to be construed as being transitory signals per se. Such transitory signals may be represented by radio waves or other freely propagating electromagnetic waves, electromagnetic waves propagating through a waveguide or other transmission media (e.g., light pulses passing through a fiber-optic cable), or electrical signals transmitted through a wire.

In some embodiments, computer-readable program instructions for systems can be downloaded to respective computing/processing devices from a computer-readable storage medium or to an external computer or external storage device via a network, such as the Internet, a local area network (LAN), a wide area network (WAN) and/or a wireless network. In some embodiments, the network may include copper transmission cables, optical transmission fibers, wireless transmission, routers, firewalls, switches, gateway computers and/or edge servers. In some embodiments, a network adapter card or network interface in each computing/processing device receives computer-readable program instructions from the network and forwards the computer-readable program instructions for storage in a computer-readable storage medium within the respective computing/processing device.

In some embodiments, computer-readable program instructions for carrying out operations of the present disclosure may be assembler instructions, instruction-set-architecture (ISA) instructions, machine instructions, machine-dependent instructions, microcode, firmware instructions, state-setting data, configuration data for integrated circuitry, or either source code or object code written in any combination of one or more programming languages, including an object-oriented programming language such as Smalltalk, C++, or the like, and procedural programming languages, such as the “C” programming language or similar programming languages.

In some embodiments, the computer-readable program instructions may execute entirely on the user's computer, partly on the user's computer, as a stand-alone software package, partly on the user's computer and partly on a remote computer or entirely on the remote computer or server. In the latter scenario, the remote computer may be connected in some embodiments to the user's computer through any type of network, including a LAN or a WAN, or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry in order to perform aspects of the present disclosure.

Embodiments of the present disclosure for detecting hypoxemia as discussed herein may be implemented using a system illustrated in FIG. 1B. Referring now to FIG. 1B, FIG. 1B illustrates an embodiment of the present disclosure of the hardware configuration of a system 30 which is representative of a hardware environment for practicing various embodiments of the present disclosure.

System 30 has a processor 31 connected to various other components by system bus 32. An operating system 33 runs on processor 31 and provides control and coordinates the functions of the various components of FIG. 1B. An application 34 in accordance with the principles of the present disclosure runs in conjunction with operating system 33 and provides calls to operating system 33, where the calls implement the various functions or services to be performed by application 34. Application 34 may include, for example, a program for detecting hypoxemia as discussed in the present disclosure, such as in connection with FIGS. 1A, 2A-2B, and 3-12.

Referring again to FIG. 1B, read-only memory (“ROM”) 35 is connected to system bus 32 and includes a basic input/output system (“BIOS”) that controls certain basic functions of system 30. Random access memory (“RAM”) 36 and disk adapter 37 are also connected to system bus 32. It should be noted that software components including operating system 33 and application 34 may be loaded into RAM 36, which may be system's 30 main memory for execution. Disk adapter 37 may be an integrated drive electronics (“IDE”) adapter that communicates with a disk unit 38 (e.g., a disk drive). It is noted that the program for detecting hypoxemia, as discussed in the present disclosure, such as in connection with FIGS. 1A, 2A-2B, and 3-12 may reside in disk unit 38 or in application 34.

System 30 may further include a communications adapter 39 connected to system bus 32. Communications adapter 39 interconnects system bus 32 with an outside network (e.g., wide area network) to communicate with other devices.

Aspects of the present invention are described herein with reference to flowchart illustrations and/or block diagrams of methods, apparatus (systems), and systems according to embodiments of the invention. It will be understood that each block of the flowchart illustrations and/or block diagrams and combinations of blocks in the flowchart illustrations and/or block diagrams can be implemented by computer-readable program instructions.

These computer-readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein includes an article of manufacture including instructions which implement aspects of the function/act specified in the flowchart and/or block diagram block or blocks. The computer-readable program instructions may also be loaded onto a computer, other programmable data processing apparatus, or other device to cause a series of operational steps to be performed on the computer, other programmable apparatus or other device to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and systems according to various embodiments of the present disclosure. In this regard, each block in the flowchart or block diagrams may represent a module, segment, or portion of instructions, which includes one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### Magnetic Resonance Devices

Additional embodiments of the present disclosure pertain to magnetic resonance devices. In some embodiments, the magnetic resonance device is optimized for measuring whole blood T_{2P} values (e.g., in a sample obtained by fingerstick, armstick or venipuncture). In some embodiments, the magnetic resonance device is operable to detect hypoxemia in a subject in accordance with the methods of the present disclosure.

In some embodiments, the magnetic resonance device includes, without limitation, a magnetic resonance relaxometry device, a table-top magnetic resonance device, a miniaturized magnetic resonance device, a benchtop magnetic resonance device, a time-domain magnetic resonance device, a magnetic resonance spectroscopy device, a magnetic resonance imaging device, a nuclear magnetic resonance device, or combinations thereof. In some embodiments, the magnetic resonance device is in the form of an instrument. In some embodiments, the magnetic resonance device is in the form of a nuclear magnetic resonance device. In some embodiments, the magnetic resonance device is in the form of a table-top miniaturized or portable magnetic resonance device.

In some embodiments, the magnet air gap and probe are reduced in diameter and optimized for small sample tubes containing human blood. In some embodiments, a cylindrical sample tube or capillary tube 3 mm or less in diameter is used. This tube geometry permits the use of magnet air gaps much smaller than typically used in commercially available magnetic resonance devices. In some embodiments, the magnet air gap and probe are optimized for non-cylindrical sample tubes, chambers or holders.

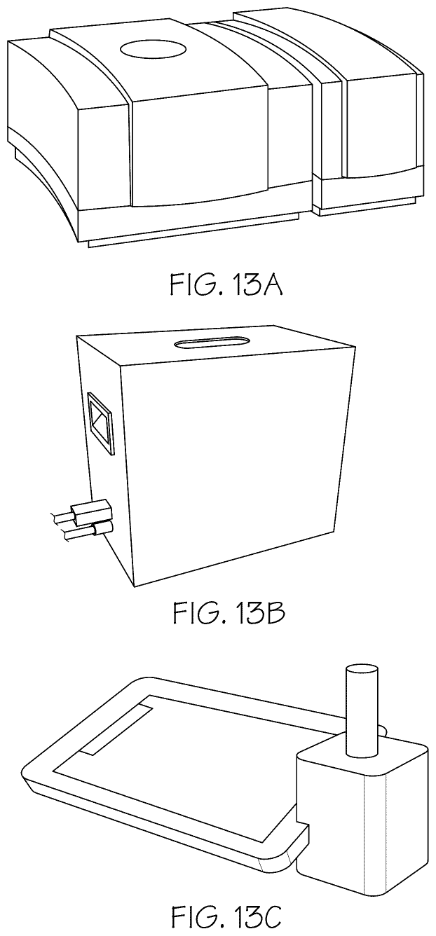

In some embodiments, the device includes a low-field permanent magnet. In some embodiments, the magnet field strength is 0.47 Tesla, corresponding to approximately 20 MHz for ¹H. In some embodiments, the magnetic field strength is approximately 0.235 Tesla, corresponding to approximately 10 MHz for ¹H. In some embodiments, the magnet field strength is approximately 1 Tesla, corresponding to approximately 42.6 MHz for ¹H. FIGS. 13A-13C illustrate additional exemplary devices.

### Additional Embodiments

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1. Whole Blood Tar Links Hemoglobin Status to Metabolic Health

Plasma and serum water T 2 , measured using benchtop magnetic resonance relaxometry, are global biomarkers of cardiometabolic health and metabolic syndrome. This Example investigated whether T₂ measured using whole blood yields similar information, as fingerstick or armstick whole blood samples would simplify metabolic health screening in point-of-care settings.

Anti-coagulated blood samples from 44 asymptomatic, non-diabetic subjects were analyzed immediately after venipuncture using a 0.47T benchtop relaxometer operating at 20 MHz for ¹H. Whole blood water T₂ values, measured using a Carr-Purcell-Meiboom-Gill pulse sequence and analyzed using discrete inverse Laplace transformation, were compared with over 100 health measures using bivariate correlation and multi-variable linear regression analyses, as well as predictor screening using a random forests or bootstrap forests machine learning algorithm.

Spontaneously-settled whole blood yielded two well-resolved components assigned to the plasma supernatant (T_{2S}) and cell pellet (T_{2P}). Paradoxically, T_{2S} correlated with hematocrit and red cell count, even though the supernatant contained no blood cells. Upon mixing whole blood with its own plasma, an inverse sixth-power dependence of T_{2S} on hematocrit was observed. This dependence was attributed to the paramagnetic relaxation enhancement of supernatant water resulting from a magnetic susceptibility difference with the paramagnetic pellet. Using this sixth-power dependence, plasma water T₂ can be estimated by extrapolation. Whole blood T_{2P} probes the state of hemoglobin inside red blood cells. Surprisingly, T_{2P} revealed correlations with markers of hyperinsulinemia, insulin resistance, oxidative stress, inflammation, adiposity, dyslipidemia, hypoxia, and cellular damage.

Water T_{2S} and T_{2P} measured in settled whole blood can detect metabolic dysregulation, early metabolic imbalance or metabolic syndrome in generally healthy subjects. The results for T_{2P} identify a novel linkage between hemoglobin oxygenation status and metabolic health. This discovery establishes the feasibility of personalized health screening using whole blood magnetic resonance relaxometry.

### Example 1.1. Human Subject Recruitment Protocol

Applicant conducted an observational cross-sectional study on 44 asymptomatic, non-diabetic human subjects who were recruited through an Institutional Review Board (IRB) protocol approved by the Institutional Review Board of the University of North Texas Health Science Center Fort Worth. The inclusion criteria were adults aged 18 and up, weighing at least 110 pounds. The exclusion criteria were diabetes (history or fasting glucose ≥125 mg/dL or A1c≥6.5), other active acute or chronic disease (history or CRP ≥10), history of bleeding disorders or difficulty donating blood, confirmed or suspected pregnancy, or not fasting at least 12 hours. All subjects were required to abstain from alcohol, exercise, over-the-counter medications, and nutritional supplements for 24 hours prior to the blood draw. The blood samples were drawn by a registered nurse-phlebotomist in the morning after an overnight fast.

### Example 1.2. NMR and Biomarker Measurements

Within 1 hour of blood collection, whole blood T₂ profiles were acquired on a Bruker mq20 Minispec (0.47T, 20 MHz for 41) using a modified Carr-Purcell-Meiboom-Gill (CPMG) pulse sequence. The sample configuration and pulse sequence are described elsewhere (J Transl Med. 2017;15(1):258. doi: 10.1186/s12967-017-1359-5.) For settled blood NMR measurements, the sample was allowed to spontaneously sediment in the 3 mm co-axial NMR insert for 40 min, and for the mixed blood, the samples were gently vortexed immediately prior to CPMG acquisition. The decay curve was analyzed with a discrete inverse Laplace algorithm (XPFit, Alango Ltd.) to extract water T₂ values.

Immediately after the phlebotomy, blood samples were processed to isolate serum and plasma for biomarker measurements. On the same day, whole blood, plasma or serum samples were sent to a CLIA-approved clinical laboratory (Atherotech or Quest) for diagnostic testing and biomarker analysis as described (J Transl Med. 2017;15(1):258. doi: 10.1186/s12967-017-1359-5.) Briefly, biomarkers of insulin and glucose metabolism, lipid and lipoprotein metabolism, inflammation, blood cells, liver, kidney and thyroid function were obtained. Whole blood and plasma viscosity were measured in-house using a VISCOLAB3000. The insulin resistance indices HOMA (homeostasis model assessment) and McAuley were calculated using the formula described in Diabetologia. 1985;28(7):412-419, and Diabetes Care. 2001;24(3):460-464, respectively.

### Example 1.3. Cell Supernatant Height and NMR Relaxation

Briefly, blood samples with different hematocrit levels were artificially generated by either adding plasma (diluting the blood cells) or removing plasma (concentrating the blood cells) from the blood samples after a brief centrifugation. Blood samples with a hematocrit as low as ˜20% and as high as ˜95% were generated by this method. The blood samples with artificially generated hematocrit were allowed to sediment before CPMG data collection, as described in the protocol for settled blood NMR relaxometry.

### Example 1.4. Data Analysis

The number of subjects (N=44) yielded a statistical power of 0.8 at a correlation coefficient of 0.4 and alpha=0.05. The decay curve acquired from each blood sample was analyzed to extract T₂ values using XPFit version 1.2.1, Alango Ltd. The decay curve for settled blood gave rise to two resolved T₂ time constants, each corresponding to supernatant and cell pellet T₂. Applicant designated these T₂ time constants as T_{2S} and T_{2P}, respectively. All other statistical analyses were performed using JMP®, Version 12.1.0 (SAS Institute Inc., Cary, NC), GraphPad Prism v. 6.05 (GraphPad Software, Inc.) and R 3.1.4 using package stats and ggplot2. The biomarker data, including in-house measurements, were log-transformed if positively skewed. Pearson' s bivariate correlations were obtained between blood water T₂ and other biomarkers. Due to occasional clinical laboratory errors in the processing of samples or sample degradation during transport, the N for a given correlation may have differed slightly.

For t-test, logistic regression and receiver operator characteristic (ROC) curve analyses, the cohort was divided into two groups of individuals having McAuley index >6.07 (No IR) and ≤6.07 (IR). Similarly, two groups of IR and No IR were defined based on the tertile criteria of six insulin resistance/glucose tolerance biomarkers (insulin, insulin c-peptide, McAuley index, triglyceride, glucose and Hb_{A1c}). In this method, the subjects were classified as insulin resistant if two or more of these six biomarkers fell in the top tertile. Student's t-test was then performed for whole blood T_{2P} in order to check whether the mean of T_{2P} was significantly different between these two groups. Finally, receiver operator characteristic (ROC) curve analyses were analyzed to compare the sensitivity and specificity of settled blood T_{2P} for detecting hyperinsulinemia/insulin resistance.

### Example 1.5. Characteristics of Human Subjects

Table 1 shows the clinical characteristics of the human subjects recruited in this Example. Overall, this is a cohort of asymptomatic adults spanning a wide age range. Although some biomarker values for specific individuals were outside the normal reference ranges, the mean values for the cohort were within those ranges. Based on Hb_{A1c} and fasting glucose values, 15 subjects had prediabetes and 29 were normoglycemic. None of the 44 subjects met the criteria for overt diabetes.

TABLE 1
Characteristics of the human study population (N = 44).

| Parameter | Mean ± SD | Range | Reference Values¹ | |
|---|---|---|---|---|
| Age | 36 ± 13 | 23-61 | n/a | |
| Gender | n/a | 20 female, 24 male | n/a | |
| Weight (kg) | 76.1 ± 19.3 | 51.7-152.0 | n/a | |
| BMI (kg/m²) | 26.2 ± 5.2 | 19.1-45.1 | >25 | (kg/m²) |
| Settled blood T_{2P} (ms) | 248.7 ± 20 | 191.7-293.0 | n.d. | |
| Settled blood T_{2S} (ms) | 719.2 ± 61.7 | 578.2-857.1 | n.d. | |
| Mixed blood T₂ (ms) | 299.7 ± 34 | 224.7-384.8 | n.d. | |
| Glucose, fasting (mg/dL) | 90.6 ± 7.6 | 71.0-109.0 | <100, non-diabetes | |
| | | | 100-125, prediabetes | |
| | | | >125, diabetes | |
| HbA_{1c} (%) | 5.5 ± 0.3 | 4.7-6.1 | <5.7, non-diabetes | |
| | | | 5.7-6.4, prediabetes | |
| | | | ≥6.5, diabetes | |
| Insulin, fasting (μIU/mL) | 8.3 ± 6.5 | 2.2-40.1 | <12.2 | |
| Insulin C-peptide, fasting (ng/mL) | 1.7 ± 0.8 | 0.7-5.1 | <2.85 | |
| Triglycerides, fasting (mg/dL) | 116.7 ± 54.1 | 50.0-276.0 | <150 | |
| hs-CRP (mg/L) | 2.4 ± 2.7 | 0.05-9.6 | <1.0, low CVD risk | |
| | | | 1.0-3.0, average risk | |
| | | | >3.0, high risk | |
| | | | ≥10, active illness | |
| WBC count (×10³/mL) | 6.6 ± 1.6 | 3.9-11.2 | 3.4-10.8 | |
| Neutrophil count (×10³/mL) | 3.5 ± 1.1 | 1.8-7.2 | 1.4-7.0 | |
| RBC count (×10⁶/mL) | 4.7 ± 0.5 | 3.8-5.8 | 4.7-6.1 | (male) |
| | | | 4.2-5.4 | (female) |

| | | | |
|---|---|---|---|
| Total cholesterol (mg/dL) | 181.6 ± 41.2 | 97.0-276.0 | <200 |
| HDL-C (mg/dL) | 52.1 ± 12.7 | 31.0-78.0 | ≥40 male, ≥50 female |
| LDL-C (mg/dL) | 107.3 ± 33.2 | 50.0-180.0 | <130 |
| Total serum protein (g/dL) | 7.1 ± 0.3 | 6.3-8.0 | 6.1-8.1² |
| Serum viscosity (cP) | 1.2 ± 0.1 | 0.9-1.4 | 1.3 ± 0.06² |
| Plasma viscosity (cP) | 1.4 ± 0.2 | 1.2-2.0 | 1.4 ± 0.08² |
| Blood viscosity (cP) | 3.1 ± 0.4 | 2.2-4.2 | 3.26 ± 0.43² |
| | | | (at shear-rate of 100 s⁻¹) |
| Thyroid stimulating hormone | 2.7 ± 1.9 | 0.01-8.31 | 0.5-4.5 |
| (mIU/mL) | | | |

¹All reference values are from Quest or Labcorp, unless noted otherwise;
²viscosity reference;
n.d., not determined;
n/a, not applicable.

### Example 1.6. Spontaneous Sedimentation and Blood Water T₂

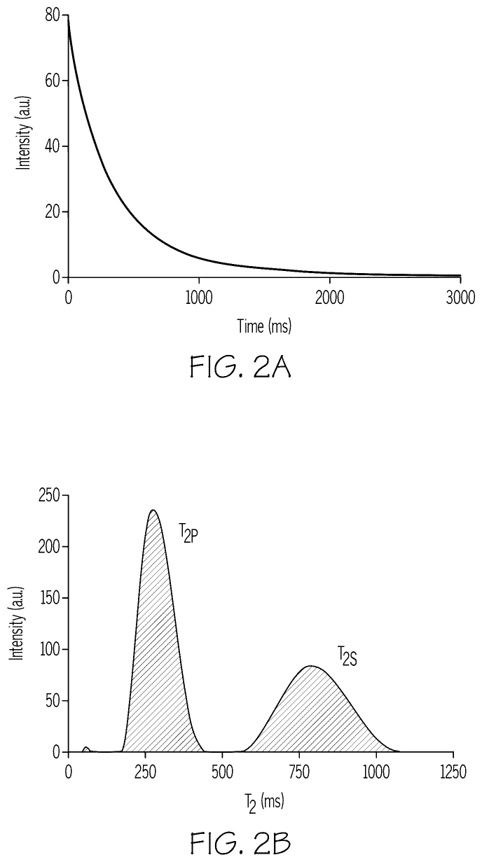

The anti-coagulated blood settled quickly and spontaneously in the NMR tube creating two separate phases. These two phases gave rise to two distinct T₂ values, T_{2S} for supernatant and T_{2P} for pellet, as shown in FIGS. 2A-2B.

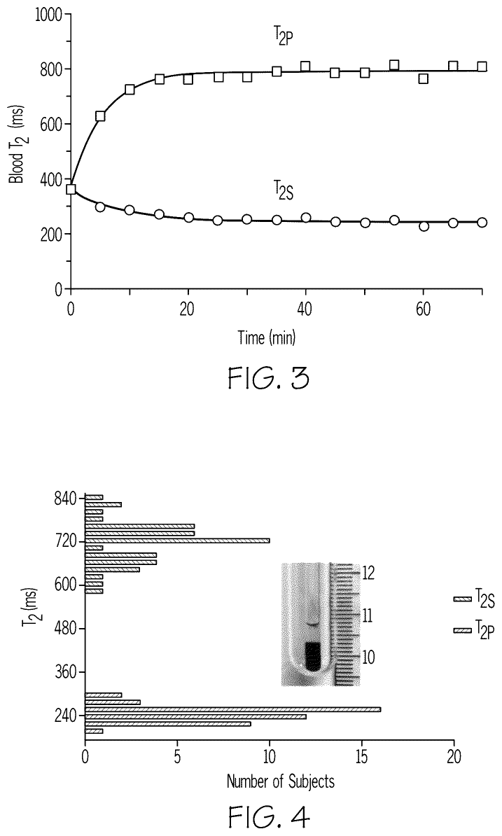

FIG. 3 shows the time-series NMR relaxation results for blood while spontaneously settling. Applicant concluded that at 40 min into sedimentation, the T₂ values for both phases had achieved equilibrium. Therefore, Applicant allowed each blood sample to settle 40 min before collecting NMR relaxation data. FIG. 4 shows the histogram of the settled blood T₂ values for the human subjects in the study cohort. The correlation between T_{2P} and T_{2S} was low (0.22) with a non-significant p-value (0.15), indicating that the values were likely uncorrelated.

### Example 1.7. Correlations of blood T₂ values with other biomarkers

Table 2 lists statistically significant correlations for pellet T_{2P}. These correlations include markers of hyperinsulinemia/insulin resistance, glucose tolerance, inflammation and dyslipidemia. Among the strongest correlations, of both negative and positive sign, were HOMA-IR, insulin C-peptide and the McAuley Index: markers of hyperinsulinemia and insulin resistance. Also, a strong correlation was observed with lactate, a marker of hypoxia. Paradoxically, the pattern of correlations was very similar to that observed for plasma water T₂, even though the blood cell pellet contains very little plasma and is dominated by packed blood cells.

TABLE 2
Bivariate correlation coefficients for T_{2P} with health measures.

| | | | P | |
|---|---|---|---|---|
| Health Measure^{a} | Huber M (95% CI) | | value | N |
| Insulin C-peptide* | −0.67 | (−0.46, −0.81) | <0.0001 | 43 |
| HOMA2-IR (C-peptide)* | −0.67 | (−0.46, −0.81) | <0.0001 | 43 |
| Complement C3c | −0.64 | (−0.41, −0.80) | <0.0001 | 40 |
| McAuley Index | +0.62 | (+0.39, +0.77) | <0.0001 | 43 |
| Apolipoprotein B | −0.60 | (−0.36, −0.76) | <0.0001 | 43 |
| LDL particle number | −0.60 | (−0.37, −0.76) | <0.0001 | 43 |
| Non-HDL-cholesterol* | −0.59 | (−0.35, −0.75) | <0.0001 | 43 |
| Alanine aminotransferase* | −0.58 | (−0.33, −0.75) | <0.0001 | 42 |
| Lactate | −0.57 | (−0.32, −0.75) | <0.0001 | 41 |
| Serum water T₂ | +0.55 | (+0.30, +0.73) | <0.001 | 43 |
| IDL-cholesterol* | −0.55 | (−0.27, −0.74) | <0.001 | 36 |
| TG/HDL ratio* | −0.54 | (−0.29, −0.72) | <0.001 | 43 |
| Immunoglobulin G | −0.53 | (−0.28, −0.72) | <0.001 | 43 |
| Body mass index* | −0.53 | (−0.28, −0.72) | <0.001 | 43 |
| LDL-cholesterol | −0.53 | (−0.27, −0.71) | <0.001 | 43 |
| Remnant-cholesterol* | −0.51 | (−0.22, −0.72) | <0.001 | 36 |
| Insulin* | −0.50 | (−0.23, −0.70) | <0.001 | 43 |
| Fibrinogen* | −0.49 | (−0.23, −0.69) | <0.001 | 43 |
| Triglycerides* | −0.48 | (−0.21, −0.68) | <0.01 | 43 |
| Serum globulins | −0.48 | (−0.20, −0.68) | <0.01 | 42 |
| Plasma globulins | −0.48 | (−0.20, −0.68) | <0.01 | 41 |
| Total cholesterol | −0.47 | (−0.20, −0.68) | <0.01 | 43 |
| Plasma water T₂ | +0.45 | (+0.17, +0.66) | <0.01 | 42 |
| HOMA2-IR (insulin)* | −0.45 | (−0.16, −0.67) | <0.01 | 39 |
| Gamma glutamyl | −0.44 | (−0.16, −0.65) | <0.01 | 43 |
| transpeptidase* | | | | |
| Lipoprotein-assoc. | +0.43 | (+0.15, +0.65) | <0.01 | 43 |
| phospholipase A₂ | | | | |
| Serum % globulins | −0.41 | (−0.12, −0.64) | <0.01 | 42 |
| Complement C4c* | −0.40 | (−0.11, −0.64) | <0.01 | 40 |
| Age* | −0.40 | (−0.11, −0.62) | <0.01 | 43 |
| VLDL-cholesterol* | −0.40 | (−0.08, −0.64) | <0.05 | 36 |
| Plasminogen activator | −0.39 | (−0.10, −0.62) | <0.05 | 43 |
| inhibitor-1* | | | | |
| Total plasma protein | −0.38 | (−0.09, −0.62) | <0.05 | 41 |
| HDL-cholesterol | +0.38 | (+0.09, +0.61) | <0.05 | 43 |
| Body temperature | −0.37 | (−0.08, −0.60) | <0.05 | 43 |
| Plasma % globulins | −0.36 | (−0.06, −0.60) | <0.05 | 41 |
| Erythrocyte sedimentation rate | −0.35 | (−0.05, −0.60) | <0.05 | 41 |
| C-reactive protein (hs-CRP) | −0.31 | (−0.01, −0.56) | <0.05 | 43 |

*these variables were natural log transformed for analysis;
†HOMA-IR (GC) is calculated from glucose and insulin C-peptide, while HOMA-IR (GI) is calculated from glucose and insulin as described in (Diabetologia. 1985; 28(7): 412-419).

Table 3 lists statistically significant correlations for supernatant T_{2S}. While some correlations expected for plasma were observed, the strongest correlations were with hematocrit and red blood cell count. That result was not anticipated.

TABLE 3
Statistically significant Pearson's product moment
correlations of settled blood T_{2S} with blood biomarkers.

| Biomarker | Pearson's correlation | P-value | N |
|---|---|---|---|
| Hematocrit | −0.608 | <0.001 | 45 |
| Red blood cell count | −0.593 | <0.001 | 45 |
| Blood viscosity 37 C. | −0.550 | <0.001 | 44 |
| Plasma total protein | −0.547 | <0.001 | 43 |
| Hemoglobin | −0.536 | <0.001 | 45 |
| IDL | −0.491 | 0.002 | 36 |
| Remnant lipoprotein | −0.485 | 0.003 | 36 |
| Plasma viscosity 37 C. | −0.482 | 0.001 | 45 |
| Total Protein | −0.474 | 0.001 | 44 |
| Albumin | −0.468 | 0.001 | 44 |
| Total LDL | −0.453 | 0.002 | 45 |
| Non HDL cholesterol | −0.443 | 0.002 | 45 |
| ApoB | −0.442 | 0.002 | 45 |
| ApoB to ApoA1 ratio | −0.436 | 0.008 | 36 |
| Total Cholesterol | −0.414 | 0.005 | 45 |
| Total VLDL | −0.392 | 0.018 | 36 |
| LDL-P | −0.383 | 0.009 | 45 |
| Triglycerides | −0.353 | 0.018 | 45 |
| HOMA IR (GC) | −0.343 | 0.021 | 45 |
| Serum viscosity 37 C. | −0.336 | 0.024 | 45 |
| Insulin c-peptide | −0.331 | 0.026 | 45 |
| Plasma albumin | −0.329 | 0.031 | 43 |
| Plasma globulin | −0.327 | 0.032 | 43 |
| Glucose | −0.309 | 0.039 | 45 |
| Fibronectin | −0.303 | 0.043 | 45 |
| Homocysteine | −0.298 | 0.047 | 45 |
| Body temperature C. | 0.304 | 0.042 | 45 |
| MCHC | 0.317 | 0.034 | 45 |
| McAuley Index | 0.362 | 0.015 | 45 |
| Chloride | 0.389 | 0.009 | 44 |
| Plasma T₂ | 0.517 | <0.001 | 44 |
| Serum T₂ | 0.557 | <0.001 | 45 |

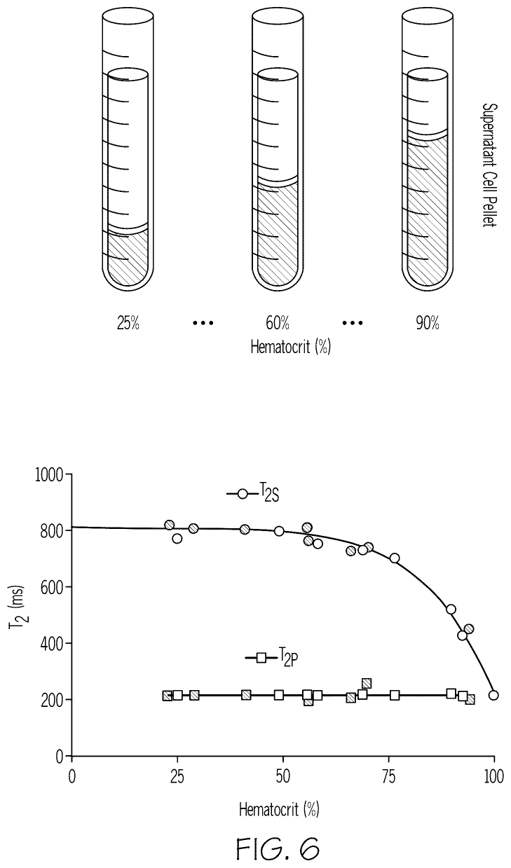

Mixed blood, as shown in Table 4, revealed only a few significant correlations, which are predominantly hemoglobin and related markers. To further investigate the unexpected association of T_{2S} with hematocrit, Applicant artificially expanded the range of hematocrit values normally observed in human blood by mixing blood cells with their own plasma, either diluting or concentrating the blood cell fraction. This permitted Applicant to examine the dependence of T_{2S} over a wider range of hematocrit values. The results are shown in FIG. 6. A steep dependence is seen at high hematocrit values, where the average distance between water in the supernatant and the pellet interface is small. The points fit well to an equation with a sixth power dependence on hematocrit. The sixth power dependence pointed to paramagnetic relaxation enhancement as the likely cause.

TABLE 4
Statistically significant Pearson's product moment
correlations of mixed blood T₂ with blood biomarkers.

| Biomarker | Pearson's correlation | P-value | N |
|---|---|---|---|
| Hemoglobin | −0.812 | <0.001 | 35 |
| Hematocrit | −0.801 | <0.001 | 35 |
| Red blood cell count | −0.634 | <0.001 | 35 |
| Blood viscosity 37 C. | −0.566 | <0.001 | 34 |
| Albumin | −0.463 | 0.006 | 34 |
| ApoB toApo-A1 ratio | −0.434 | 0.024 | 27 |
| Albumin to globulin ratio | −0.376 | 0.028 | 34 |
| Homocysteine | −0.363 | 0.032 | 35 |
| Ceruloplasmin | 0.347 | 0.041 | 35 |
| Platelet count | 0.384 | 0.023 | 35 |
| Cortisol | 0.412 | 0.014 | 35 |
| ESR | 0.435 | 0.011 | 33 |
| Alpha-1 antitrypsin | 0.589 | <0.0010 | 35 |

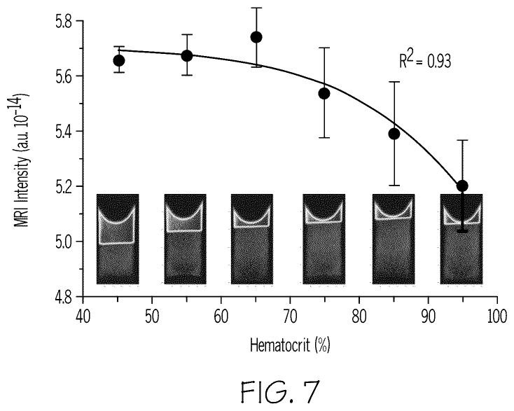

To further assess the paramagnetic relaxation enhancement (PRE) effect, Applicant collected T₂-weighted images using a benchtop MRI instrument and analyzed the signal intensities of slices through the supernatant. As shown in FIG. 7, the intensities also revealed a sixth-power dependence on hematocrit, although the precision of the MRI intensity measurements was not nearly as good as the precision of the quantitative T₂ measurements from benchtop relaxometry.

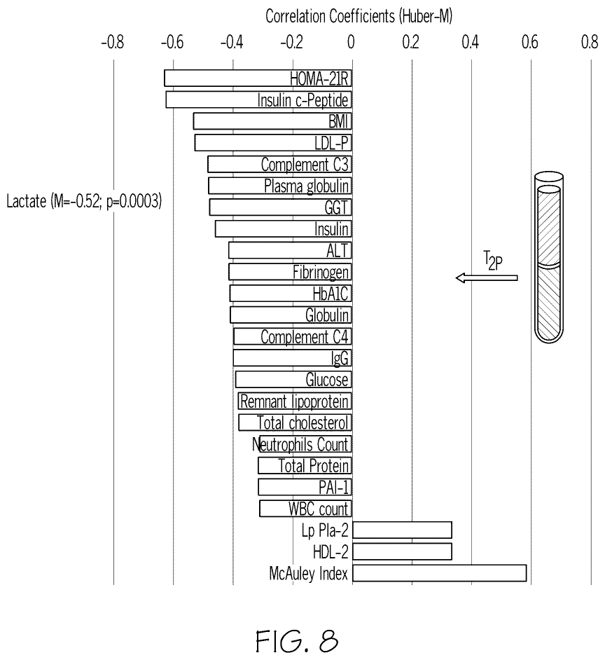

FIG. 8 provides a summary of correlation coefficients (Huber M-values) for T_{2P} and biomarkers of cardiometabolic health. These observations were unexpected, as T_{2P} monitors hemoglobin status. Confidence intervals and p-values are not shown. However, all of the correlation coefficients in FIG. 8 were statistically significant (p<0.05).

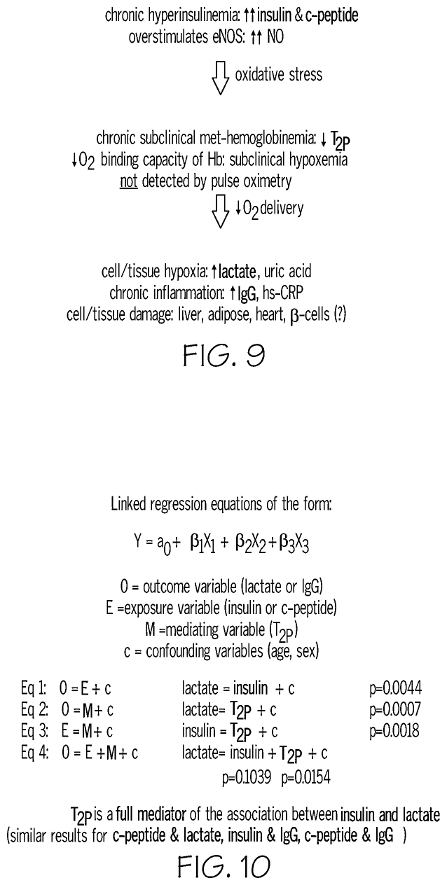

FIG. 9 provides a hypothetical mechanistic link whereby hemoglobin oxidation (subclinical met-hemoglobinemia) mediates the association between hyperinsulinemia and downstream hypoxia and chronic inflammation. FIG. 10 illustrates a causal mediation analysis to test the hypothesis in FIG. 9, using four (4) linked regression equations. The variables highlighted in red were statistically significant in the linked equations. The observed p-values for lactate as the outcome variable, insulin as the exposure and T_{2P} as the mediator, are listed.

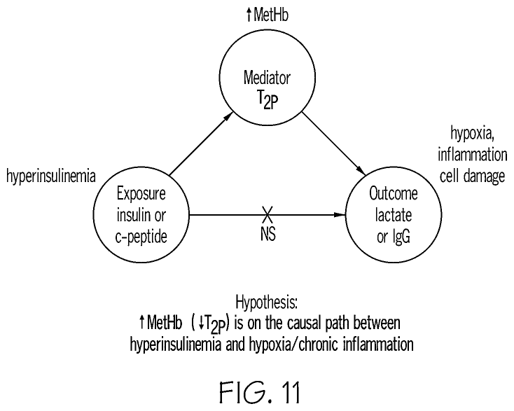

FIG. 11 provides a schematized representation of the results reported in FIG. 10. When the regression equation includes the mediator (T_{2P}), the association between exposure and outcome is non-significant (NS).

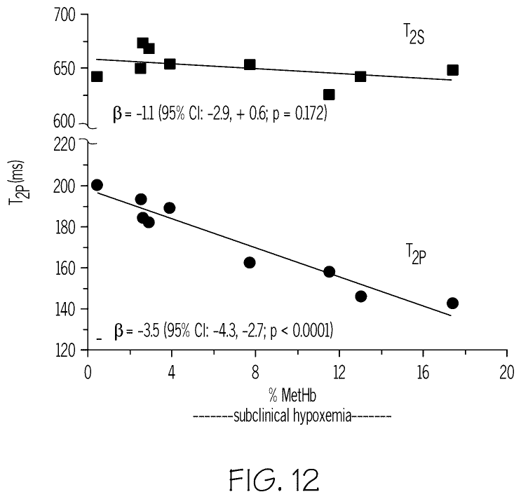

FIG. 12 shows the association between T_{2P} and % MetHb in a whole blood sample titrated with sodium nitrite, a pro-oxidant. This compound increased hemoglobin oxidation, thereby increasing % Met Hb and decreasing T_{2P}. FIGS. 13A-13C show examples of benchtop, portable or miniaturized magnetic resonance relaxometry devices that can be used to measure T_{2S} and T_{2P} in whole blood.

In sum, settled whole blood yields two T₂ values: (1) T_{2S}, which represents plasma water T₂ after correcting for (Hct)⁶; and (2) T_{2P}, which monitors the oxygenation and oxidation state of Hb. Surprisingly, T_{2P} was also correlated with markers of cardiometabolic health in apparently healthy adults. As such, T_{2P} also links hemoglobin status to cardiometabolic health. Without being bound by theory, the causal mediation analysis supports the hypothesis that a lower T_{2P} value likely represents a higher Met-Hb value, which mediates the association of hyperinsulinemia with hypoxia and chronic inflammation.

### Example 1.8. Discussion

Human blood is a complex biofluid, and water protons are distributed in several microenvironments within blood, such as protons in the intracellular fluid and plasma, including water protons that are hydrogen-bonded to proteins and other molecules.

In settled anti-coagulated whole blood, at least two distinct water proton microenvironments can be resolved in the time domain, yielding two distinct T₂ values. The higher value is designated T_{2S}, as it arises from the plasma supernatant. The lower value is T_{2P}, as it arises from the blood cell pellet. The NMR signal intensity from a blood sample (e.g., a pellet of the blood sample) is predominately from red blood cells, as red blood cells outnumber white blood cells and platelets by approximately 1000 to 1. The most abundant source of protons (hydrogens) in red blood cells is water molecules, with smaller contributions from proteins, especially hemoglobin, and membrane lipids. Thus, T_{2P} is predominately a measure of the T₂ relaxation time constant of water molecules inside red blood cells.

Oxygen-carrying hemoglobin is dissolved in the intracellular water of red blood cells. The water molecules bind to hemoglobin, generating an equilibrium between bound and unbound water, and a kinetic exchange of water on and off the protein. The observed T_{2P} value arises from the weighted average of bound and unbound water, as the on/off exchange rate is fast compared with the T₂ time scale. Thus, water molecules are intimately associated with the concentrated hemoglobin inside red blood cells, and can sense changes in its oxygenation and oxidation state, as well as its concentration.

Hemoglobin that lacks bound oxygen (deoxy-hemoglobin or deoxy-Hb) is paramagnetic, as the unoccupied heme groups have unpaired electrons. The paramagnetic heme lowers the observed T_{2P} value through a phenomenon called paramagnetic relaxation enhancement. By contrast, hemoglobin molecules with bound oxygen (oxy-hemoglobin or oxy-Hb) have no unpaired electrons, are not paramagnetic, and do not cause paramagnetic relaxation enhancement. The observed T_{2P} value for oxy-hemoglobin is higher than that for deoxy-hemoglobin. In this manner, T_{2P} is sensitive to the oxygenation state of reduced (functional) hemoglobin.

When hemoglobin becomes oxidized to form MetHb, it can no longer bind oxygen, but instead binds water molecules at the heme site where oxygen normally binds. Since MetHb is paramagnetic, the T_{2P} is further lowered by paramagnetic relaxation enhancement, which is strengthened by the very close proximity of the water bound at the heme site. Thus, T_{2P} is highly sensitive to the presence and amount of oxidized or MetHb as a percentage of total hemoglobin.

A preliminary ex vivo experiment with whole human blood supports proof-of-concept (FIG. 12). Samples of anticoagulated whole human blood from a healthy human subject were titrated with increasing amounts of 0.2% sodium nitrite, an oxidizing agent. Whole blood T_{2P} was measured on each sample, and the state of the hemoglobin was measured using blood gas analysis. As the concentration of MetHb (as percent of total hemoglobin) increased from 0.4% (no sodium nitrite added) to 17.4% (40 μadded), the T_{2P} value decreased in a linear fashion from 199.7±0.6 msec to 142.8±0.6 msec. Across the range of added sodium nitrite, the percentage of reduced, deoxy-hemoglobin remained low, <1%. Thus, the T_{2P} value decreased with increasing amounts of MetHb in the blood, across the range expected for subclinical hypoxemia and subclinical methemoglobinemia in humans.

In principle, the NMR relaxation of the protons in different microenvironments gives rise to different T₂ relaxation constants as shown in equation 1.

$$I ( t ) = {\sum }_{i} {A}_{i} {e}^{- t / {T}_{2 i}} \qquad ( 1 )$$

Here, I(t) is nuclear magnetic resonance (NMR) signal intensity, Aᵢ is signal amplitude and T₂ᵢ is the transverse relaxation constant of ith proton microenvironment or mobility domain

In mixed blood, red blood cells are suspended in the plasma, and water protons are in chemical exchange between the intracellular and extra-cellular compartments of mixed blood. The rate of chemical exchange is fast on the NMR T₂ time scale, giving rise to one observed T₂ component that is the weighted average between the two compartments. However, in settled blood, the water protons are not able to undergo fast chemical exchange between the two compartments because of the physical barriers created by the packed cells and the longer diffusion distance. This generates two distinct, slowly exchanging populations of water protons in the pellet and plasma supernatant. Therefore, settled blood, in contrast to mixed blood, gives two T₂ values, one each for plasma and cell pellet. In spectroscopy experiments, this phenomenon has also been reported to give rise to two separate NMR resonances for water protons in settled blood, implying slow exchange on the chemical shift time scale. Within the cell pellet, the protons in the intracellular and extracellular environments are in fast exchange, with the extracellular pool within the packed pellet being sparsely populated with water compared with the intracellular pool. This gives rise to only one observed T₂ for the pellet.

The paradoxical observation of strong correlations between T₂ and hematocrit, over the narrow range of hematocrit normally found in human blood, was puzzling at first. However, the in vitro generation of samples with artificially high hematocrit revealed the steep dependence shown in FIG. 6. In turn, the observed sixth power dependence on hematocrit pointed to paramagnetic relaxation enhancement (PRE) as the likely mechanism, as PRE has a theoretical six-power distance dependence.

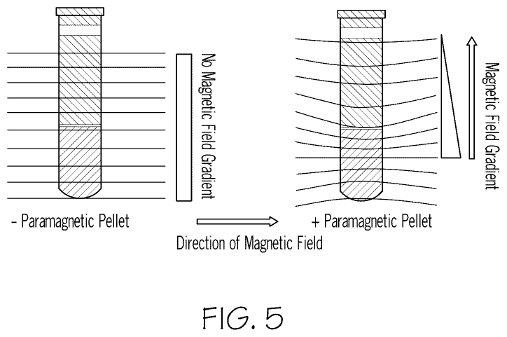

Hemoglobin exists in the oxygenated and deoxygenated forms, with the former being diamagnetic, and the latter, paramagnetic. Hemoglobin in blood exposed to room air includes a small percentage of deoxy hemoglobin and hence, is paramagnetic. By contrast, the plasma supernatant is essentially diamagnetic. As illustrated in FIG. 5, this difference creates a magnetic susceptibility gradient between the pellet and supernatant, which falls off as the sixth power of the distance from the pellet. As the hematocrit increases, the height of the supernatant becomes smaller, and the average distance between the rapidly diffusing water molecules in the supernatant and the pellet becomes smaller. Hence, water molecules in a short supernatant experience more of the magnetic susceptibility gradient. This gradient results in greater paramagnetic relaxation enhancement and shorter T_{2S} values at higher hematocrit. In practice, the PRE effect can be exploited to assess hematocrit and red blood cell count in T₂ measurements of settled whole blood.

In contrast, the pellet T_{2P} did not show the expected correlations with hematocrit and red blood cell count but revealed strong correlations with markers of insulin resistance/hyperinsulinemia, inflammation, dyslipidemia, oxidative stress and cellular damage (i.e., the pathophysiology of metabolic syndrome). This observation establishes a linkage between hemoglobin status (measured by T_{2P}) and metabolic health. Without being bound by theory, Applicant hypothesizes that T_{2P} is detecting subclinical hypoxemia and possible tissue hypoxia.

The strong correlations of T_{2S} with metabolic biomarkers, as shown in Table 2, indicate that settled whole blood is surprisingly informative and provides a practical sample format for assessing metabolic health by NMR relaxometry. Small amounts of whole blood can be obtained by fingerstick or armstick, without the need for phlebotomy and centrifugation, which opens up the possibility of performing whole blood NMR relaxometry in point-of-care settings such as clinics.

In sum, this Example describes a method for detecting hypoxemia, such as in situations where hypoxemia is least expected (e.g., in asymptomatic, apparently healthy individuals). The method records the spin-spin relaxation time constant of the cell pellet (T_{2P}) in a sample of settled, anti-coagulated whole human blood. The measurement is made using a compact tabletop magnetic resonance relaxometry device. The small blood sample can be obtained using conventional venipuncture or from a fingerstick drop. Whole blood T_{2P} reports on the oxygenation and oxidation state of hemoglobin, a key protein inside red blood cells that delivers oxygen from the lungs to vital organs and tissues.

A low T_{2P} value in an otherwise healthy individual points to a hidden impairment that could slowly and insidiously deprive cells of oxygen, resulting in cell and tissue damage. Specific examples could include slow damage to the oxygen-dependent insulin-secreting beta cells of the pancreas, leading to type 2 diabetes, or the cells lining the interior wall of arteries, leading to cardiovascular disease. Whole blood T_{2P} overcomes the limitations of pulse oximetry and the impracticality of blood gas analysis for routine health screening and monitoring. It addresses an unmet need for better tools for the early detection of hidden health conditions in order to prevent diabetes, prediabetes, cardiovascular disease, metabolic syndrome and related conditions.

In a recent study of 44 asymptomatic, generally healthy adults, Applicant observed that T_{2P} values varied over a surprisingly wide range, indicating varying degrees of hemoglobin oxygen binding and/or oxidation. Moreover, the low T_{2P} values were associated with elevated markers of insulin resistance/hyperinsulinemia, inflammation, oxidative stress and cellular damage. For the first time, this discovery links hemoglobin status with metabolic health. Whole blood T_{2P} solves the problem of undetected subclinical hypoxemia and gives clinicians an innovative new tool for early detection and disease prevention.

Whole blood T_{2P} uses a different approach for probing the status of hemoglobin distinct from pulse oximetry and blood gas analysis. Whole blood T_{2P} directly monitors the interior of red blood cells, which contain a highly concentrated aqueous solution of hemoglobin. Whole blood T_{2P} monitors the hydrogen nuclei in the abundant water molecules, as well as those in the Hb. T_{2P} can readily detect the difference between oxy-hemoglobin (hemoglobin saturated with bound oxygen) and deoxy-hemoglobin (hemoglobin without bound oxygen). Deoxy-Hb is paramagnetic, whereas oxy-Hb is not. The paramagnetism lowers the ¹H T_{2P} value through a process known as paramagnetic relaxation enhancement. Likewise, T_{2P} can detect the difference between oxidized MetHb and non-oxidized oxy-Hb, as only the former is paramagnetic. Since MetHb binds water, not oxygen, the key probe of hemoglobin status is located right at the site of the oxidized heme group. Paramagnetic relaxation enhancement is maximized when the ¹H probe is closest to the paramagnetic center.

Thus, whole blood T_{2P} responds in a sensitive manner to changes in the amount of MetHb inside red blood cells. The measured T_{2P} reflects the combined presence of deoxy-Hb and MetHb: the lower the T_{2P}, the higher the levels of deoxy-Hb and/or MetHb. As both forms of hemoglobin decrease the delivery of oxygen to tissues, whole blood T_{2P} can detect subclinical hypoxemia that is missed by conventional pulse oximetry.

Another novel element of this method is that the measurement is made in a small volume of settled whole human blood. Therefore, the blood cells need not be separated from the liquid plasma, making this method practical for point-of-care analysis, as well as for conventional clinical lab analysis of blood samples obtained using venipuncture. Finally, this technology includes a novel table-top magnetic resonance device optimized for measuring whole blood T_{2P}.

T_{2P} can be measured using existing relaxometry devices, even though they were not designed nor optimized for this purpose. Currently, Applicant is using a Bruker mq20 relaxometer to measure T_{2P} in blood. In particular, Applicant is using a Bruker mq20 relaxometer to measure T_{2P} in a 40 microliter sample of settled whole blood. The sample has an upper plasma supernatant and lower cell pellet. The sample is contained in a 3 mm-diameter coaxial insert housed within an empty 10 mm outer tube. The magnet air gap, probe size and instrument are much larger than needed, and the experiment is less efficient than it would be using a device optimized for this purpose.

To date, Applicant has established correlations between T_{2P} and over 120 markers of human health status in 44 individuals. These results established that T_{2P} can be measured in whole human blood and established the linkage between hemoglobin status (T_{2P}) and metabolic health.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of detecting hypoxemia in a subject, said method comprising:
receiving a blood sample from the subject;
measuring the T₂ relaxation time constant of the blood cells of the blood sample (T_{2P} value); and
correlating the measured T_{2P} value to hypoxemia.

2. The method of claim 1, wherein the T_{2P} value represents the spin-spin relaxation time constant of the blood cell component of the blood sample, wherein the blood cell component is in the form of a pellet of an anti-coagulated whole blood sample.

3. The method of claim 1, wherein the T_{2P} value is represented in the following formula:
$$I ( t ) = {\sum }_{i} {A}_{i} {e}^{- t / {T}_{2 i}}$$
wherein I(t) represents the nuclear magnetic resonance (NMR) signal intensity, Aᵢ represents the signal amplitude, and T₂ᵢ represents the transverse relaxation time constant of ith proton microenvironment or mobility domain

4. The method of claim 1, wherein the correlating comprises correlating a lower-than-average T_{2P} value to hypoxemia.

5. The method of claim 4, wherein the lower-than-average T_{2P} value is attributed to elevated levels of deoxy hemoglobin (deoxy-Hb), elevated levels of oxidized hemoglobin (MetHb), or combinations thereof.

6. The method of claim 1, further comprising a step of correlating the measured T_{2P} value to the subject's susceptibility to one or more hypoxemia-related conditions.

7. The method of claim 6, wherein the one or more hypoxemia-related conditions is selected from the group consisting of hypoxia, cellular damage, tissue damage, type 2 diabetes, cardiovascular disease, hyperinsulinemia, insulin resistance, inflammation, oxidative stress, dyslipidemia, hyperglycemia, early metabolic imbalance, prediabetes, metabolic syndrome, adiposity, or combinations thereof.

8. The method of claim 6, wherein a lower-than-average T_{2P} value is correlated to the subject's susceptibility to one or more hypoxemia-related conditions.

9. The method of claim 1, further comprising a step of making a treatment decision.

10. The method of claim 9, wherein the treatment decision comprises monitoring the subject for signs or symptoms of hypoxemia, administering a therapeutic agent to the subject, or combinations thereof.

11. The method of claim 9, wherein the method further comprises implementing the treatment decision.

12. The method of claim 9, wherein the method is repeated after implementing the treatment decision.

13. The method of claim 1, wherein the subject is a human being.

14. The method of claim 1, wherein the subject shows no hypoxemia-related symptoms.

15. The method of claim 1, wherein the method occurs manually.

16. The method of claim 1, wherein the method occurs through the utilization of a computer program.

17. A system for detecting hypoxemia in a subject, wherein the system comprises one or more computer-readable storage mediums having a program code embodied therewith, wherein the program code comprises programming instructions for:
receiving a blood sample from the subject;
measuring the T₂ relaxation time constant of blood cells of the blood sample (T_{2P} value); and
correlating the measured T_{2P} value to hypoxemia.

18. The system of claim 17, wherein the programming instructions correlate a lower-than-average T_{2P} value to hypoxemia.

19. The system of claim 17, wherein the system further comprises programming instructions for correlating the measured T_{2P} value to the subject's susceptibility to one or more hypoxemia-related conditions.

20. The system of claim 19, wherein the programming instructions correlate a lower-than-average T_{2P} value to the subject's susceptibility to one or more hypoxemia-related conditions.

21. The system of claim 17, wherein the system further comprises programming instructions for recommending a treatment decision.

22. The system of claim 17, wherein the system is in the form of a web-based program, an application-based program, or combinations thereof.
